(19) United States
(12) Patent Application Publication
(10) Pub. No.: US 2014/0220646 A1
(43) Pub. Date: Aug. 7, 2014

(54) APPARATUS FOR TREATING FOOD WASTE AND EXTRACTING BIO OIL

(71) Applicant: Won Im Lim, Gyeonggi-do (KR)

(72) Inventor: Won Im Lim, Gyeonggi-do (KR)

(73) Assignee: G&P BIOTECH CO., LTD., Seoul (KR)

(21) Appl. No.: 14/240,546

(22) PCT Filed: Apr. 26, 2012
(86) PCT No.: PCT/KR2012/003206

§ 371 Date: Apr. 10, 2014

(30) Foreign Application Priority Data
Aug. 24, 2011 (KR) 10-2011-0084323

(51) Int. Cl. C12M 1/00; C11B 13/00

(52) U.S. Cl. CPC C12M 47/10; C11B 13/00
USPC 435/134; 435/290.2

(57) ABSTRACT

An apparatus for treating food waste and extracting bio oil includes: a fermentation dryer which ferments and decomposes food waste for 24 hours to reduce moisture contained in the food waste to 40 to 50%, a crusher which crushes the food waste into small particles, a distillation tank which heats the crushed food waste at low pressure and temperature so that oil is gasified by means of low temperature thermal decomposition to form oil gas, a distillation tower in which the oil gas is cooled to be liquefied and separated into moisture (carbon liquid fertilizer) and oil, a vacuum storage tank which collects the separated oil, and a centrifugal separator which rectifies the oil. The apparatus extracts a carbon liquid fertilizer, bio oils, and activated carbon while treating organic substances such as food waste and residual product compost.

### BACKGROUND OF THE INVENTION

The present invention relates to an apparatus and method of using the apparatus for treating organic waste such as food waste and extracting bio oil, and more particularly to an apparatus for treating organic waste such as food waste and extracting bio oil which is configured such that food waste is introduced into a fermentation dryer and fermentation chamber (referred to as a “fermentation dryer”), in which microorganisms are present, and then fermented, dried, and decomposed for 24 hours so as to be reduced in weight, the resultant dried. and decomposed organic substances are crushed into small particles using a crusher, the crushed particles are heated at low pressure and temperature within a distillation tank so that moisture and oil components can be gasified by means of a low temperature thermal decomposition, the separated and discharged gas is cooled and liquefied in a distillation tower so that moisture (carbon liquid fertilizer, also known as liquid carbon fertilizer and liquid carbon-based fertilizer) and oil are separated during the liquefaction, the separated oil is collected in a vacuum storage tank, and the collected oil is supplied to a centrifugal separator so that the oil can be refined. The apparatus of the present invention can extract a liquid fertilizer (carbon fertilizer), bio oils, and activated carbon (carbon) while treating organic substances such as food waste, residual product compost, etc.

Conventionally, in order to treat food waste, various treatment methods such as composting, drying, fermentation drying, and carbonization, and transformation into feed have been developed and widely spread so far, and are still under development.

Among them, the most common conventional food treatment methods are long-period fermentation drying, and burning-and-carbonization. The conventional food waste treatment methods do not include an idea of extracting oils from food waste.

### SUMMARY OF THE INVENTION

Accordingly, the present invention has been made keeping in mind the above problems occurring in the related art, and is intended to provide an apparatus for treating organic waste such as food waste and extracting bio oils, which is configured such that food waste is introduced into a fermentation dryer and fermentation chamber (referred to as a “fermentation dryer”), in which microorganisms are present, and then fermented, dried, and decomposed for 24 hours so as to be reduced in weight, the resultant dried and decomposed organic substances are crushed into small particles using a crusher, the crushed particles are heated at low pressure and temperature within a distillation tank so that vapor and oil can be gasified by means of a low temperature thermal decomposition, the separated and discharged gas is cooled and liquefied in a distillation tower so that moisture (carbon liquid fertilizer) and oil are separated during the liquefaction, the separated oil is collected in a vacuum storage tank, and the collected oil is supplied to a centrifugal separator so that the oil can be refined. The apparatus can extract a liquid fertilizer (carbon liquid fertilizer), bio oils, and activated carbon (carbon) while treating organic substances such as food waste, residual product compost, and the like.

According to one aspect, the apparatus includes:

a fermentation dryer equipped with a mixing chamber into which a bulking agent and microorganisms are preliminarily introduced and in which a mixing vane, which slowly mixes food waste is installed, an oxygen supplying apparatus, and an air cleaning apparatus are installed, wherein the fermentation dryer ferments and decomposes food waste for 24 hours while mixing the food waste, thereby reducing moisture contained in the food, waste to 40% to 50%, resulting in a decrease in the weight of the food waste;

a crusher which crushes the food waste and organic substances which are deprived of a portion of moisture into small particles and supplies the crushed food waste and organic substances to a distillation tank using a screw conveyer;

the distillation tank including a heating chamber under which a burner is installed, and a tank having an agitator which rotates by driving force of a motor and agitates organic particles therein and an discharge hole formed at an upper end thereof and connected to a distillation tower, wherein the distillation tank heats the organic particles supplied from the crusher through the discharge hole at low pressure and temperature so that vapor and oil gas are sequentially obtained by means of low temperature thermal decomposition;

the distillation tower in which first through third coolant pipes through which coolant is circulated by a compressor are installed and in which the vapor and oil gas supplied from the distillation tank are cooled to be liquefied by coming into contact with the first to third coolant pipes, the distillation tower being equipped with a liquid fertilizer valve at a lower portion thereof, which controls discharge of liquid fertilizer;

a plurality of oil valves installed at different heights to discharge oil stored in the distillation tower;

a plurality of oil transportation pipes which transports liquefied oil to oil tanks via glass tubes when each of the oil valves is open;

the oil tanks which temporarily store oil;

a vacuum storage tank which stores the oil supplied from each of the oil tanks;

vacuum pumps which create a vacuum pressure in the vacuum storage tank so that an inside space of the distillation tank becomes low in pressure; and

a centrifugal separator which rectifies the oil supplied from the vacuum storage tank through a centrifugal process.

According to the present invention, it is possible to extract a carbon liquid fertilizer, bio oils, and activated carbon (carbon) by fermenting and drying food waste, crushing a mixture supplied from a fermentation chamber, and performing distillation under reduced pressure on the crushed particles. Accordingly, it is possible to prevent the environment from being polluted. In addition, since odors and gases generated in an extraction process are completely removed, the present invention contributes to prevention of environmental pollution.

### BRIEF DESCRIPTION OF THE DRAWINGS

FIG. 1 is a diagram illustrating an apparatus for treating food waste and extracting bio oil according to the present invention; and

FIG. 2 is a flowchart illustrating a process of extracting bio oils according to the present invention.

### DETAILED DESCRIPTION OF THE INVENTION

An apparatus according to an embodiment of the present invention is structured such that food waste is introduced into a fermentation dryer, in which microorganisms are present, and then fermented, dried, and decomposed for 24 hours to be reduced in weight, the resultant dried and decomposed organic substances are crushed into small particles using a crusher, the crushed particles are heated at low pressure and temperature within a distillation tank so that vapor and oil can be gasified by means of low temperature thermal decomposition, the separated and discharged gas is cooled and liquefied in a distillation tower so that moisture (carbon liquid fertilizer) and oil are separated during the liquefaction.

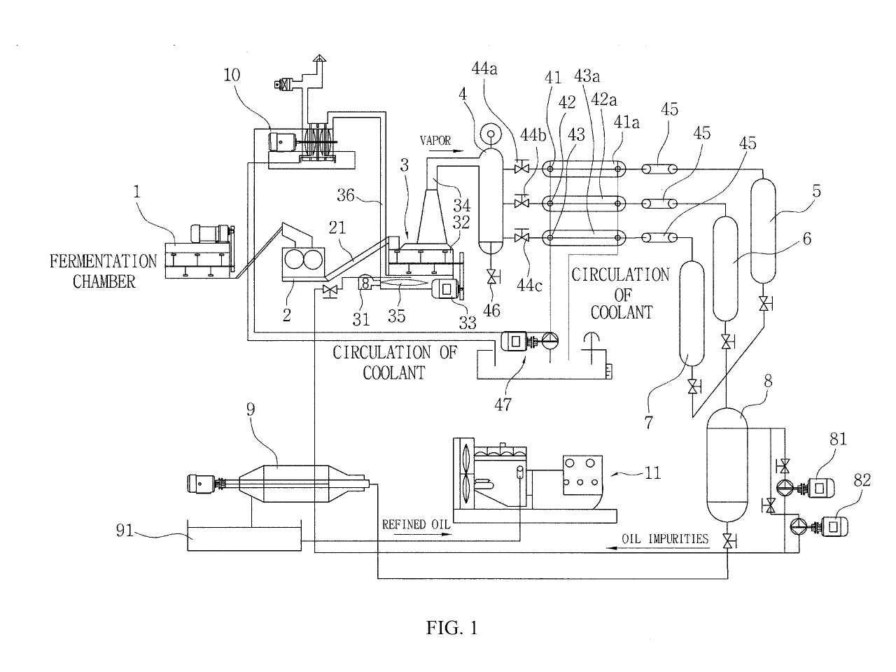

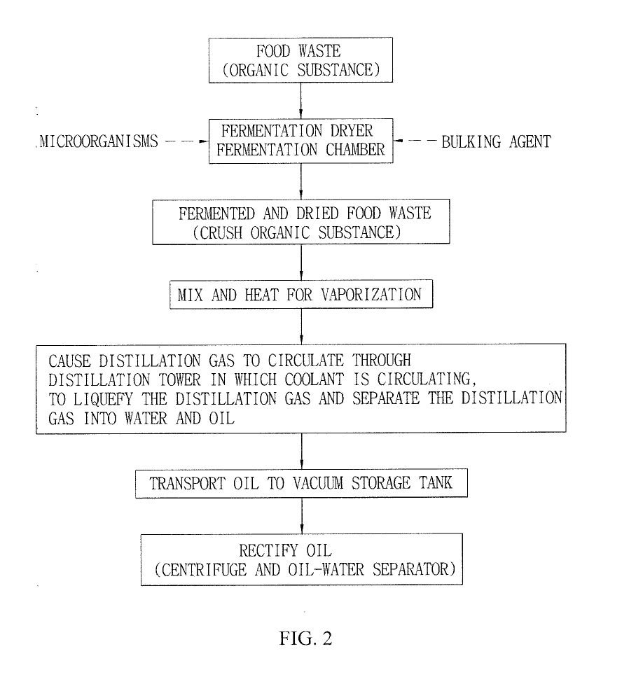

Hereinbelow, a preferred embodiment of the present invention will be described with reference to attached FIGS. 1 and 2.

With reference to FIGS. 1 and 2, an apparatus for treating food waste and extracting bio oil includes:

a fermentation dryer 1 equipped with a mixing chamber into which a bulking agent and microorganisms are preliminarily introduced and in which a mixing vane, which slowly mixes food waste is installed, an oxygen supplying apparatus, and an air cleaning apparatus are installed, wherein the fermentation dryer ferments and decomposes food waste for 24 hours while mixing the food waste, thereby reducing moisture contained in the food waste to 40% to 50%, resulting in a decrease in the weight of the food waste;

a crusher 2 which crushes the food waste and organic substances which are deprived of a portion of moisture in the fermentation dryer 1 into small particles and supplies the crushed food waste and organic substances to a distillation tank 3 using a screw conveyer 21;

the distillation tank 3 including a heating chamber 35 under which a burner 31 is installed, and a tank having an agitator 32 which rotates by driving force of a motor 33 and agitates organic particles and a discharge opening 34 formed at an upper end thereof and connected to a distillation tower 4, wherein the distillation tank 3 heats the organic particles supplied from the crusher 2 through the discharge opening at low pressure and temperature so that vapor and oil gas are sequentially obtained by means of low temperature thermal decomposition;

the distillation tower 4 in which first through third coolant pipes 41 to 43 through which coolant is circulated by a compressor 47 are installed, and in which the vapor and oil gas supplied from the distillation tank 3 are cooled to be liquefied by coming into contact with the first to third coolant pipes 41 to 43, the distillation tower 4 being equipped. with a liquid fertilizer valve 46 at a lower portion thereof, which controls discharge of a liquid fertilizer;

a plurality of oil valves 44a to 44c installed at different heights to discharge oil stored in the distillation tower 4;

a plurality of oil transportation pipes 41a to 43a which transports liquefied oil to oil tanks 5 to 7 via glass tubes 8 when each of the oil valves 44a to 44c is open;

the oil tanks 5 to 7 which temporarily store oil;

a vacuum storage tank 8 which stores the oil supplied from each of the oil tanks 5 to 7;

vacuum pumps 81, 82 which create a vacuum (i.e., sub-atmospheric) pressure in the vacuum storage tank 8 so that an inside space of the distillation tank 3 becomes low (i.e., sub-atmospheric) pressure; and

a centrifugal separator 9 which rectifies the oil supplied from the vacuum storage tank 8 through a centrifugal process.

A supply pump 82 which pumps and supplies impurities and gases contained in oil to the heating chamber 35 of the distillation tank 3 is communicates with a top portion of the vacuum storage tank 8 and also functions as a vacuum pump for the vacuum storage tank 8 as stated hereinabove. The impurities floating on the oil in the vacuum storage tank 8 are burned by the burner 31 and removed.

The fermentation dryer 1 ferments and decomposes food waste and compost to reduce a moisture content of the food waste and compost to 40 to 50%.

In the mixing chamber of the fermentation dryer 1, a bulking agent and microorganisms to ferment food waste are preliminarily introduced. In addition, the mixing vane to mix the food waste is also installed in the mixing chamber. The mixing vane is rotated by driving force of a motor. The oxygen supplying apparatus and air cleaning apparatus are installed so that the oxygen supplying apparatus supplies oxygen necessary for fermentation to the mixing chamber, and the air cleaning apparatus removes odor generated during the fermentation.

When the food waste, microorganisms, and bulking agent are introduced into the fermentation dryer 1 and then fermented and dried for 24 hours, most of the food waste is decomposed and residuals such as starch, seeds, and bones containing moisture of 40 to 50% remain.

That is, the food waste is reduced in weight after it is fermented and dried, and the organic substances such as starch, seeds, and bones which are not decomposed by the microorganisms remain.

This fermentation dryer can be installed and used in apartment complexes, housing complexes, service food facilities, restaurants, etc.

The crusher 2 crushes the food waste which is discharged after the fermentation from the fermentation dryer 1 into small particles.

The fermented and dried food waste is introduced into the crusher 2 to be crushed, and the crushed particles are supplied to the distillation tank 3 via the screw conveyer 21.

The distillation tank 3 heats the small organic particles supplied from the crusher 2 at low pressure and temperature so that the organic particles are thermally decomposed. During this process, vapor (carbon liquid fertilizer) and oil components are sequentially gasified, and activated carbon (carbon) is obtained at the last stage.

The distillation tank 3 includes the heating chamber 38 under which the burner 31 is installed. The agitator 32 is installed in the distillation tank 3 and is rotated by driving force of the motor 33 to agitate the organic particles. The distillation tank 3 has the discharge opening 34 formed at an upper end thereof and connected to the distillation tower 4. The organic particles supplied from the crusher 2 are heated at a low temperature to undergo low temperature thermal decomposition so that vapor and oil gases are sequentially generated and discharged with change in temperatures.

Since the distillation tank 3 is at low pressure because a vacuum pressure is created by the vacuum pumps 82, thermal decomposition is performed at low pressure.

When the inside of the distillation tank 3 is heated by heat of the burner 31, the organic powder introduced into the distillation tank 3 starts to be decomposed, moisture is gasified first and then discharged through the discharge opening 34 and supplied to the distillation tower 4. After moisture is almost gasified, oil components in the organic powder are gasified and then discharged through the discharge opening 34 and supplied to the distillation tower 4. After moisture and oil components all evaporate, activated carbon (carbon) remains in the distillation tank 3 so that activated carbon (carbon) can be obtained.

The distillation tower 4 is a tower to liquefy vapor or oil supplied. from the distillation tank 3. The first to third coolant pipes 41 to 43 through which coolant is circulated by the compressor 47 are installed in the distillation tower 4.

Accordingly, when vapor and oil gases, which are produced through gasification in the distillation tank 3, are introduced into the distillation tower 4, the vapor and oil gases are cooled and liquefied while coming into contact with the first to third coolant pipes 41 to 43 so that water and oil collect in the distillation tower 4.

In this case, the gas supplied. from the distillation tank mostly contains liquid fertilizer components at an early stage of gasification and is liquefied to be stored in the distillation tower 4. The gas supplied from the distillation tank 3 at a later stage mostly contains oil components and is liquefied.

Accordingly, the carbon liquid fertilizer obtained through the liquefaction at the early stage is collected by opening the liquid fertilizer valve 46 installed at a lower portion of the distillation tower 4, and the liquefied oil stored in the distillation tower 4 can be discharged through the oil transportation. pies 41a to 43a by opening the oil valves 44a to 44c installed, at different heights on the side surface of the distillation tower 4.

Even though liquid fertilizer and oil exist in a mixed state in the distillation tower 4, since the liquid fertilizer and oil are different in specific gravity, the oil may float on the liquid fertilizer. Accordingly, the oil and liquid fertilizer can be separately extracted.

The glass tubes 45 are connected to rear ends of the of transportation pipes 41a to 43a, respectively. The glass tubes 45 allow an operator to inspect the state of the transported oil. Accordingly, management and maintenance of instruments is easy.

The oils transported through the glass tubes 45 are stored in the oil tanks 5 to 7, and the oil stored in each of the oil tanks 5 to 7 can be transported to the vacuum storage tank 8 by control of the valves.

The oil transported to the vacuum storage tank 8 is supplied to the centrifugal separator 9 and undergoes a centrifugal process to be rectified. The rectified oil is discharged to the storage tank 91. In this way, oil of high quality can be obtained,

Reference number 11 refers a generator which generates electricity using the oil stored in the storage tank 91 as raw material. That is, it is possible to generate electricity from oil extracted from food waste.

Impurities contained in oil stored in the vacuum storage tank 8 float in the vacuum storage tank 8, and these floating impurities are continuously supplied to the heating chamber 35 of the distillation tank 3 to be burned and removed by the burner 31.

Reference number 10 refers a desulfurizer. The desulfurizer 10 removes sulfur from waste gas exhausted through a chimney 36 connected to the heating chamber 35 using a water cooling system.

1. An apparatus for treating food waste and extracting bio oil, comprising:
a fermentation dryer equipped with a mixing chamber into which a bulking agent and microorganisms are preliminarily introduced and in which a mixing vane which slowly mixes food waste is installed, an oxygen supplying apparatus, and an air cleaning apparatus, wherein the fermentation dryer ferments and decomposes food waste for 24 hours while mixing the food waste, thereby reducing moisture contained in the food waste to 40% to 50%, resulting in a decrease in the weight of the food waste;
a crusher which crushes the food waste and organic substances which are deprived of a portion of moisture in the fermentation dryer into small particles and supplies the crushed food waste and organic substances to a distillation tank using a screw conveyer;
the distillation tank including a heating chamber under which a burner is installed, and a tank equipped with an agitator which rotates by driving force of a motor and agitates organic particles is installed and a discharge opening formed at an upper end of the tank and connected to a distillation tower, wherein the distillation tank heats the organic particles supplied from the crusher through the discharge opening at low pressure and temperatures so that vapor and oil gas are sequentially obtained by means of low temperature thermal decomposition;
the distillation tower in which first through third coolant pipes through which coolant is circulated by a compressor are installed, and the vapor and oil gas supplied from the distillation tank are cooled to be liquefied by coming into contact with the first to third coolant pipes, the distillation tower being equipped with a liquid fertilizer valve at a lower end thereof, which controls discharge of a liquid fertilizer;
a plurality of oil transportation pipes which transport liquefied oil to oil tanks via a glass tube when each of the oil valves is open;
oil tanks which temporarily store oil;
a vacuum storage tank which stores the oil supplied from each of the oil tanks;
vacuum pumps which create a vacuum pressure in a vacuum storage tank so that an inside space of the distillation tank becomes sub-atmospheric in pressure; and
a centrifugal separator which rectifies the oil supplied from the vacuum storage tank through a centrifugal process.

2. The apparatus for treating food waste and extracting bio oil, according to claim 1, further comprising:
a supply pump which pumps and supplies impurities and gases contained in oil to the heating chamber of the distillation tank and which communicates with the top portion of the vacuum storage tank, wherein the impurities floating on the oil in the vacuum storage tank are burned by the burner and thereby disposed of.

3. The method of using the apparatus of claim 1 to treat food waste and extract bio oil.
